(19) United States
(12) Patent Application Publication
(10) Pub. No.: US 2018/0169328 A1
(43) Pub. Date: Jun. 21, 2018

(54) DRUG MONITORING DEVICE FOR INTRAVENOUS INFUSION AND THE METHOD THEREOF

(71) Applicant: Industrial Technology Research Institute, Hsinchu (TW)

(72) Inventors: Cheng-Yi Yang, Hsinchu (TW); Yi-Fei Luo, Hsinchu (TW)

(21) Appl. No.: 15/430,744

(22) Filed: Feb. 13, 2017

(30) Foreign Application Priority Data
Dec. 21, 2016 (TW) 105142461

(51) Int. Cl. A61M 5/168; G01N 15/14; G01F 23/292; G01F 23/00

(52) U.S. Cl. CPC A61M 5/16831; G01N 15/1459; G01F 23/2921; A61M 2205/331; G01N 15/1431; A61M 2205/18; G01F 23/0007

(57) ABSTRACT

The disclosure provides a drug monitoring device assembled outside an intravenous infusion set for intravenous infusion, including a fastening assembly, a monitoring sensor mounted on the fastening assembly, and a monitoring module. The monitoring sensor includes at least one turbidity light transmitter for emitting first light beams and at least one light receiver. The first light beams change in directions when passing through the intravenous infusion set. The at least one light receiver generates a first sensing signal in response to receiving one of the first light beams. The monitoring module generates a turbidity warning message when a number of the first sensing signals received during an estimation time period is greater than a turbidity warning threshold.

### CROSS-REFERENCE TO RELATED APPLICATIONS

The present application is based on, and claims priority from, Taiwan Application Number 105142461, filed on Dec. 21, 2016. The disclosure of which is hereby incorporated by reference herein in its entity.

### TECHNICAL FIELD

The disclosure is directed to a drug monitoring device for intravenous infusion and the method thereof.

### BACKGROUND

In clinical medicine, the need of intravenous infusion admixture often occurs. For example, antibiotics are mostly powder; therefore, the antibiotics require to be mixed with normal saline or dextrose solution before injecting into a patient. Because the limitation on the infusion amount of a patient, it is necessary for mixing drugs when the patient needs to be injected with variety of drugs.

Sometime there may be no drug-drug interaction or adverse drug reactions. However, the intravenous infusion admixture of drug may cause particle coagulation, condense, and turbid in the intravenous infusion set. If the size of particles for the infusion medicine is too large, the particles may accumulate in human's organs such as heart, lung, liver, kidney, muscle, skin, capillary bore, vessel and so on. This may cause diseases like blood clot thrombosis, venous hypertension, pulmonary hypertension, idiopathic pulmonary fibrosis or even cancer.

The timing and the position of chemical reaction of mixed drugs may vary according to liquid density and medication characteristics of drugs. In order to avoid turbidity of intravenous infusion, the liquid needs to be detected in real time and monitored continuously. However, there are a variety of drugs, it is usually difficult to identify the occurrence of turbidity or precipitation after mixing drugs by naked eyes. Therefore, the medical risks increase when the turbidity of intravenous infusion is detected and monitored by the artificial judgment.

### SUMMARY

In one of the exemplary embodiments, the disclosure is directed to a drug monitoring device assembled to an intravenous infusion set for intravenous infusion. The drug monitoring device for intravenous infusion comprises a fastening assembly assembled on the intravenous infusion set, a monitoring sensor mounted on the fastening assembly, and a monitoring module. The monitoring sensor comprises at least one turbidity light transmitter for emitting first light beams and at least one light receiver. The first light beams change in directions when passing through the intravenous infusion set. The at least one light receiver receives a part of the first light beams, which change in directions, and generates a first sensing signal in response to receiving one of the first light beams. The monitoring module is configured to receive the first sensing signal, and generate a turbidity warning message when a number of the first sensing signals received during an estimation time period is greater than a turbidity warning threshold.

In one of the exemplary embodiments, the disclosure is directed to a drug monitoring method applied to a drug monitoring device for intravenous infusion. The drug monitoring device assembled outside an intravenous infusion set comprises at least one turbidity light transmitter, at least one light receiver and a monitoring module. The method comprises: emitting first light beams by the at least one turbidity light transmitter, wherein the first light beams change in directions when passing through the intravenous infusion set; receiving a part of the first light beams with changes in directions by the at least one light receiver; generating a first sensing signal by the at least one light receiver in response to receiving one of the first light beams; recording a number of the first sensing signals received by the monitoring module during an estimation time period; and generating a turbidity warning message by the monitoring module when the number of the first sensing signals received during the estimation time period is greater than a turbidity warning threshold.

The foregoing will become better understood from a careful reading of a detailed description provided herein below with appropriate reference to the accompanying drawings.

### BRIEF DESCRIPTION OF THE DRAWINGS

FIG. 1 is a block diagram illustrating an exemplary embodiment of a drug monitoring device for intravenous infusion, according to the disclosure.

FIG. 2 is a block diagram illustrating another exemplary embodiment of a drug monitoring device for intravenous infusion, according to the disclosure.

FIG. 3 is a block diagram illustrating another exemplary embodiment of a drug monitoring device for intravenous infusion, according to the disclosure.

FIG. 4 is a block diagram illustrating another exemplary embodiment of a drug monitoring device for intravenous infusion, according to the disclosure.

FIG. 5 illustrates an exemplary embodiment of the drug monitoring device for intravenous infusion, according to the disclosure.

FIG. 6 shows a schematic view illustrating how an intravenous infusion set is clamped by a drug monitoring device, according to an exemplary embodiment of the disclosure.

FIG. 7A is a schematic drawing illustrating a top view of a drug monitoring device, according to an exemplary embodiment of the disclosure.

FIG. 7B is a schematic drawing illustrating a cross section of a plurality of turbidity light transmitters that monitor turbidity, according to an exemplary embodiment of the disclosure.

FIG. 8A is a schematic drawing illustrating a top view of a drug monitoring device, according to another exemplary embodiment of the disclosure.

FIG. 8B is a schematic drawing illustrating a cross section of a plurality of liquid-level light transmitters that detect a liquid level, according to another exemplary embodiment of the disclosure.

FIG. 9 illustrates a flow chart of a monitoring drug method for intravenous infusion, according to an exemplary embodiment of the disclosure.

FIG. 10 illustrates another physical structure of the drug monitoring device for intravenous infusion, according to an exemplary embodiment of the disclosure.

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

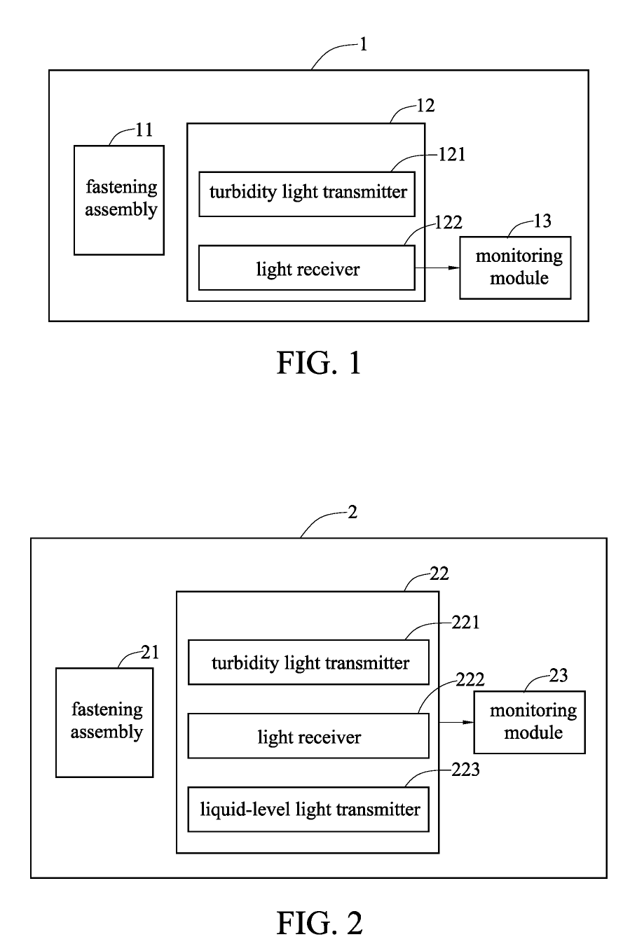

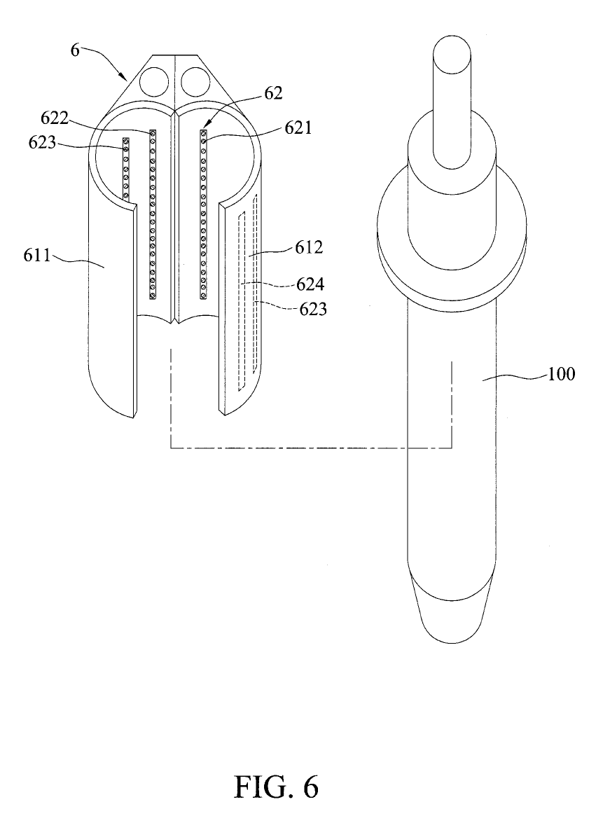

The disclosure is directed to a drug monitoring device for intravenous infusion and a method thereof. FIG. 1 is a block diagram illustrating an exemplary embodiment of a drug monitoring device for intravenous infusion, according to the disclosure. Referring to FIG. 1, the exemplary embodiment of a drug monitoring device 1 for intravenous infusion is adapted to be assembled outside an intravenous infusion set (shown in FIG. 6) for monitoring a turbidity in the intravenous infusion set. The drug monitoring device 1 comprises a fastening assembly 11, a monitoring sensor 12 and a monitoring module 13.

The fastening assembly 11 is assembled on the intravenous infusion set (shown in FIG. 6) so that the drug monitoring device 1 may combined with the intravenous infusion set. In this disclosure, the fastening assembly 11 may be fixed on the intravenous infusion set, for example, by the way of clamping, locking or attaching.

The monitoring sensor 12, mounted on the fastening assembly 11, comprises at least one turbidity light transmitter 121 and at least one light receiver 122. The at least one turbidity light transmitter 121 and the light receiver 122 are mounted on an inner surface of the fastening assembly 11 (that is, the surface facing to the intravenous infusion set), wherein the light receiver 122 is arranged at a monitoring angle with respect to the at least one turbidity light transmitter 121.

The at least one turbidity light transmitter 121 steadily emits first light beams, and the first light beams pass through the intravenous infusion set (shown in FIG. 6). The turbid particles in the intravenous infusion set will cause the first light beams to change in directions when the first light beams pass through the intravenous infusion set. Then, part of the first light beams changed in directions will be received by the at least one light receiver 122. In response to receiving one of the first light beams, the at least one light receiver 122 generates a first sensing signal.

The monitoring angle decides that the at least one light receiver 122 receives the first light beams which only within a certain angle range. For example, when the monitoring angle is 90 degrees, that is to say, when the two lines, from the at least one turbidity light transmitter 121 and the at least one light receiver 122 to a center of the intravenous infusion set (shown in FIG. 6), form a right angle, the at least one light receiver 122 receives the first light beams which change in directions around 90 degrees. The more the first light beams that the at least one light receiver 122 receives, the more turbid particles are in the intravenous infusion set, so the amount of first light beams received by the at least one light receiver 122 may reflect the turbidity in the intravenous infusion set. The monitoring angle may be, but not limited to 90 degrees. The monitoring angle may be set based on different measurement technologies.

The number of the at least one turbidity light transmitter 121 and the at least one light receiver 122 may vary according to different design considerations for different sizes or types of intravenous infusion set (shown in FIG. 6). The at least one turbidity light transmitter 121 and the at least one light receiver 122 in this disclosure may be, for example, infrared transmitter and infrared receiver, respectively, and the first light beams have a wavelength that can pass through liquid.

The monitoring module 13 is configured to receive the first sensing signal and to generate a turbidity warning message only when the number of the first sensing signals received during an estimation time period is not less than a turbidity warning threshold. The higher the turbidity in the intravenous infusion set is, the more degrees the first light beams change in directions. Therefore, when the monitoring module 13 receives the first sensing signal and the number of the first sensing signal received during the estimation time period (for example, 1 second) exceeds the turbidity warning threshold, it means that the turbidity in the intravenous infusion set (shown in FIG. 6) is getting higher and then the monitoring module 13 alarms people around by generating the turbidity warning message.

In one of the exemplary embodiments, to monitor the whole intravenous infusion set (shown in FIG. 6), the number of the at least one turbidity light transmitter 121 and the at least one light receiver 122 are both more than one. The turbidity light transmitters 121 are arranged in a line, for example in a striped line or in a direction perpendicular to a horizontal plane. The light receivers 122 that correspond to the turbidity light transmitters 121 may be correspondingly arranged in a line as well so that the liquid of different heights in the intravenous infusion set may be monitored at the same time. The turbidity is estimated according to the number of the first sensing signals received by the light receivers 122, and the intravenous infusion need to be stopped using when the mixed drugs are mismatched and this cause the turbidity in the intravenous infusion set is, for example, over 0.5 NTU (Nephelometric Turbidity Unit).

FIG. 2 is a block diagram illustrating another exemplary embodiment of a drug monitoring device for intravenous infusion, according to the disclosure. Referring to FIG. 2, the another exemplary embodiment of a drug monitoring device 2 for intravenous infusion is adapted to be assembled outside an intravenous infusion set (shown in FIG. 6) for monitoring a turbidity in the intravenous infusion set. The drug monitoring device 2, similar to the drug monitoring device shown in FIG. 1, comprises a fastening assembly 21, a monitoring sensor 22 and a monitoring module 23. The drug monitoring device 2 may monitor both the turbidity and liquid level in the intravenous infusion set, therefore, the monitoring sensor 22 of the drug monitoring device 2 further comprises at least one liquid-level light transmitter 223 mounted on the fastening assembly 21. As shown in FIG. 2, the monitoring sensor 22 comprises at least one turbidity light transmitter 221, at least one light receiver 222 and the at least one liquid-level light transmitter 223.

The at least one liquid-level light transmitter 223 is used to steadily emit second light beams, and the second light beams may or may not pass through the intravenous infusion set (shown in FIG. 6). The at least one light receiver 222 of the monitoring sensor 22 may or may not receive the second light beams. The intravenous infusion set may or may not contain liquid. When there is liquid in the intravenous infusion set, the liquid in the intravenous infusion set will block the second light beams or cause the second light beams to change in directions. The at least one light receiver 222 of the monitoring sensor 22 generates a second sensing signal in response to receiving one of the second light beams, and then the monitoring module 23 determines a liquid level in the intravenous infusion set based on a sensing result of the at least one light receiver 222. In other words, there is liquid in the intravenous infusion set when the monitoring module 23 receives any second sensing signal from the at least one light receiver 222.

In an embodiment, the monitoring module 23 may determine that there is no liquid of a certain height in the intravenous infusion set when receiving a second sensing signal corresponding to the certain height. In the other hand, the monitoring module 23 may determine that there is liquid of a certain height in the intravenous infusion set when without receiving any second sensing signal corresponding to the certain height. Therefore, position information of the light receiver 222 in the drug monitoring device 2 and position information of the intravenous infusion set (shown in FIG. 6) with respect to the drug monitoring device 2 may be pre-defined, so the monitoring module 23 may estimate the liquid level by using the second sensing signal and the pre-defined position information of both the light receiver 222 and the intravenous infusion set.

In one of the exemplary embodiments, the at least one liquid-level light transmitter 223 and the at least one light receiver 222 are located on two opposite sides of the intravenous infusion set (shown in FIG. 6), respectively, to have the second light beams passing through the intravenous infusion set. That is to say, the center of the intravenous infusion set is right on the connecting line between at least one liquid-level light transmitter and the at least one light receive 222. In this disclosure, the second light beams may be, for example, infrared light, that is, which have a wavelength that cannot pass through liquid. The at least one turbidity light transmitter 221 emits first light beams, and the liquid-level light transmitter 223 emits second light beams, so the light receiver 222 may sense both the first light beams and the second light beams to monitor the turbidity and the liquid level in the intravenous infusion set.

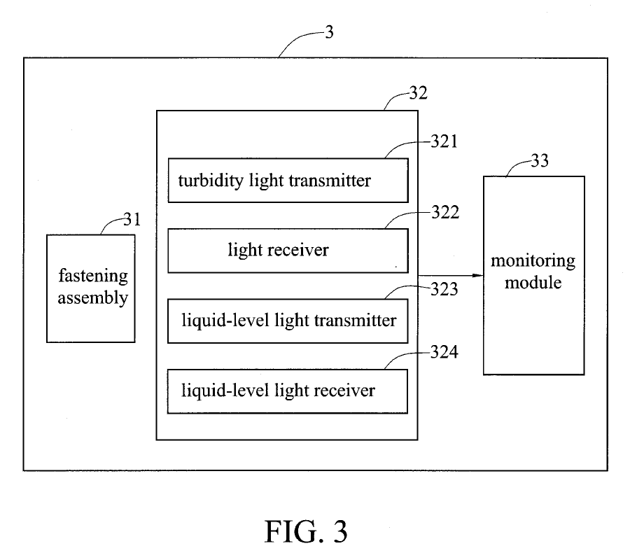

FIG. 3 illustrates a drug monitoring device 3 for intravenous infusion according to another exemplary embodiment of the disclosure, which is similar to the one in FIG. 1 but comprises another embodiment of a monitoring sensor 32. Similar to the exemplary embodiment of FIG. 1, the monitoring sensor 32 may monitor the turbidity in the intravenous infusion set (shown in FIG. 6), and comprises at least one turbidity light transmitters 321, and at least one light receiver 322. The monitoring sensor 32 shown in FIG. 3, may further monitor the liquid level in the intravenous infusion set as well, wherein the monitoring sensor 32 further comprises at least one liquid-level light transmitter 323, and at least one liquid-level light receiver 324.

The at least one liquid-level light transmitter 323 is used to steadily emit second light beams, and the second light beams may or may not pass through the intravenous infusion set (shown in FIG. 6). When there is liquid in the intravenous infusion set, the liquid in the intravenous infusion set will block the second light beams or cause the second light beams to change in directions. The at least one liquid-level light receiver 324 generates a second sensing signal in response to receiving one of the second light beams, and then a monitoring module 33 of the drug monitoring device 3 determines a liquid level in the intravenous infusion set based on a sensing result of the at least one liquid-level light receiver 324.

In one of the exemplary embodiments, the at least one liquid-level light transmitter 323 and the at least one liquid-level light receiver 324, both mounted on inner surface of a fastening assembly 31 of the drug monitoring device 3 (i.e. the surface facing to the intravenous infusion set), are located on two opposite sides of the intravenous infusion set (shown in FIG. 6), respectively. That is to say, the center of the intravenous infusion set is right on a connecting line between at least one liquid-level light transmitter 323 and the at least one liquid-level light receive 324.

In an embodiment, the monitoring module 33 may determine that there is no liquid of a certain height in the intravenous infusion set when receiving a second sensing signal corresponding to the certain height. In the other hand, the monitoring module 33 may determine that there is liquid of a certain height in the intravenous infusion set when without receiving any second sensing signal corresponding to the certain height. Therefore, position information of the liquid-level light receiver 324 in the drug monitoring device 3 and position information of the intravenous infusion set (shown in FIG. 6) with respect to the drug monitoring device 3 may be pre-defined, so that the monitoring module 33 may estimate the liquid level by using the second sensing signal and the pre-defined position information of both the liquid-level light receiver 324 and the intravenous infusion set.

The monitoring module 33 compares the liquid level with a liquid-level threshold, and generates a liquid-level warning message to alarm people around when the liquid level is lower than the liquid-level threshold.

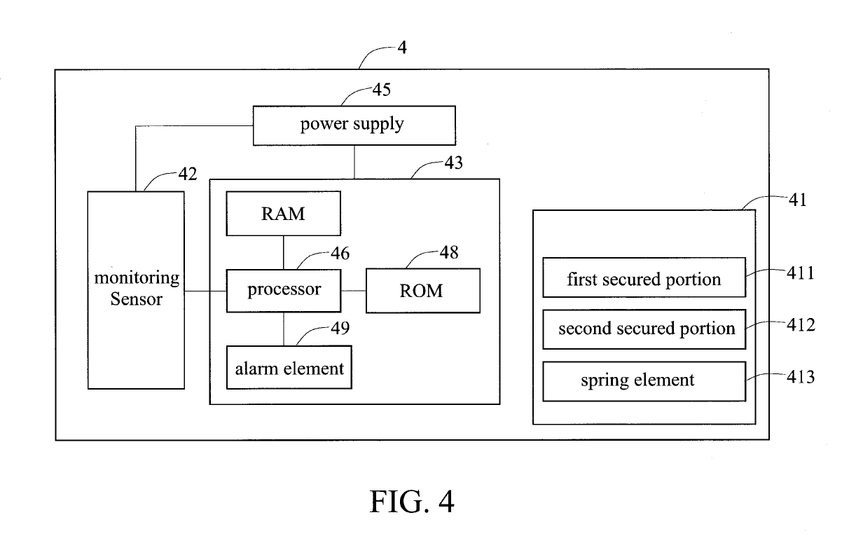

FIG. 4 is a block diagram illustrating another exemplary embodiment of a drug monitoring device for intravenous infusion, according to the disclosure. Referring to FIG. 4, a monitoring module 43 of a drug monitoring device 4 is realized by a processor 46, a ROM 48 and an alarm element 49. The ROM 48 stores program codes executed by the processor 46 which receives sensing signals from a monitoring sensor 42. The alarm element 49 is triggered by the processor 46 to alarm people around by such as light, sound, visual picture, text and so on. The alarm element 49 may be, but not limited to buzzer, speaker, vibrator or LED (Light Emitting Diode). The drug monitoring device 4 further comprises a power supply 45 (for example, battery).

A fastening assembly 41 of the drug monitoring device 4 is installed on the intravenous infusion set (shown in FIG. 6). In one of exemplary embodiments, the fastening assembly 41 comprises a first secured portion 411, a second secured portion 412 coupled to the first secured portion 411, and a spring element 413. The spring element 413 is clamped between the first secured portion 411 and the second secured portion 412 so that the first secured portion 411 and the second secured portion 412 elastically clamping each other around the intravenous infusion set. The fastening assembly 41 may be designed to attach to the intravenous infusion set, for example, by a screw lock or by a rotation positioning member.

In one of exemplary embodiments, the fastening assembly 41 may be designed as two block entities crossover to each other. One of the block entities combined with a spring is steadily pushing against the other block entity, and users may pull the block entity combined with the spring slightly away from the other one and put the intravenous infusion set between the two entities so that the intravenous infusion set may be attached to the drug monitoring device 4.

In other words, the aforesaid clamping scheme to have the drug monitoring device being firmly fastened on the intravenous infusion set may be rotationally clamped or elastically clamped. Also, the fastening scheme may be, but not limited to a clamping scheme.

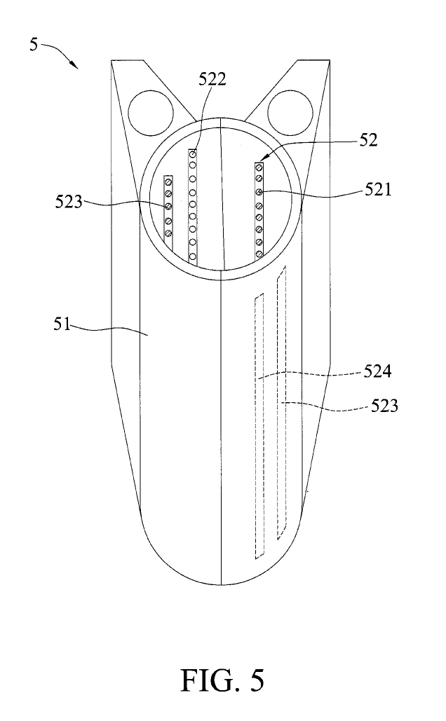

FIG. 5 illustrates an exemplary embodiment a monitoring sensor 52 of a drug monitoring device 5 for intravenous infusion, according to the disclosure. Turbidity light transmitter 521, liquid-level light transmitter 522, light receivers 523 and liquid-level light receiver 524 are arranged in vertical stripes, respectively, on an inner surface of a fastening assembly 51 of the drug monitoring device 5.

FIG. 6 shows a schematic view illustrating how an intravenous infusion set 100 is clamped by a first secured portion 611 and a second secured portion 612 of a drug monitoring device 6, according to an exemplary embodiment of the disclosure. When the drug monitoring device 6 is combined with the intravenous infusion set 100, turbidity light transmitter 621, liquid-level light transmitter 622, light receivers 623 and liquid-level light receiver 624 of a monitoring sensor 62 are positioned on an outer surface of the intravenous infusion set 100.

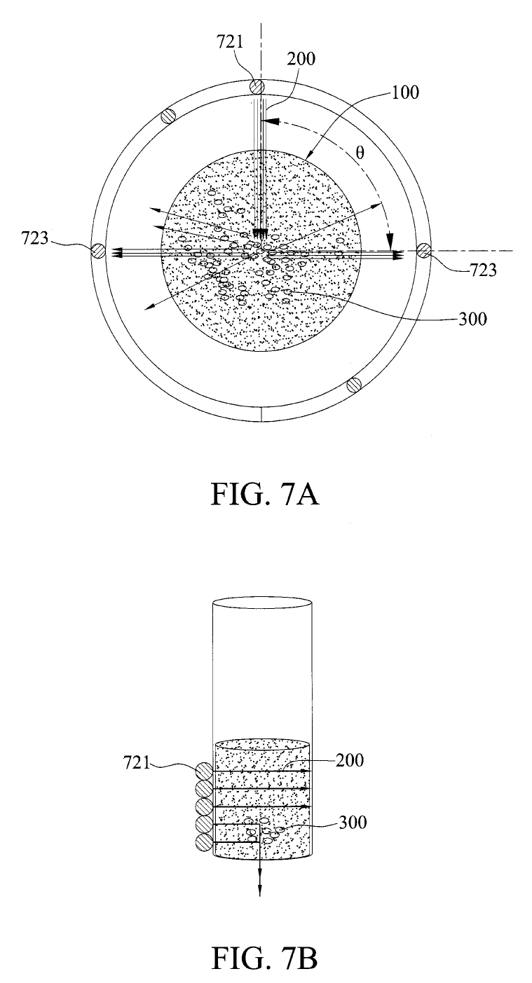

FIG. 7A and FIG. 7B are two schematic drawings illustrating a top view of a drug monitoring device and a cross section of turbidity light transmitters that monitor turbidity, respectively, according to an exemplary embodiment of the disclosure. Referring to FIG. 7A and FIG. 7B, a turbidity light transmitter 721 and light receivers 723 are positioned around an intravenous infusion set 100. A monitoring angle θ between the two lines respectively from the turbidity light transmitter 721 and one of the light receivers 723 to the center of intravenous infusion set 100 is 90 degrees. The monitoring angle θ may be set based on different measurement technologies, and may be, but not limited to 90 degrees.

In one of exemplary embodiments under working conditions, first light beams 200, emitted from the turbidity light transmitter 721, change in directions due to reflection or refraction when there are turbid particles 300 in the intravenous infusion set 100, so that the light receivers 723 on two sides may receive part of the first light beams 721 and generate a first sensing signal in response to receiving the part of the first light beams. In the other hand, the first light beams 200 will pass through the intravenous infusion set 100 if the liquid inside is clear. To monitor the turbidity based on number of first light beams 200 which change in directions, the light receivers 723 on two sides need to avoid being arranged right in the connecting line between the turbidity light transmitter 721 and the central axis of the intravenous infusion set 100.

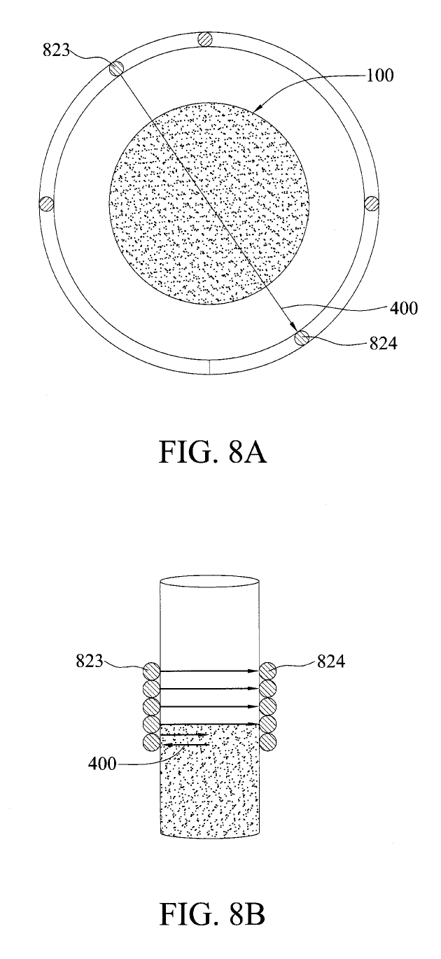

FIG. 8A and FIG. 8B are two schematic drawings illustrating a top view of a drug monitoring device and a cross section of liquid-level light transmitters that detect a liquid level, respectively, according to another exemplary embodiment of the disclosure. Referring to FIG. 8A and FIG. 8B, liquid-level light transmitters 823 and liquid-level light receivers 824, positioned on an outer surface of an intravenous infusion set 100, are arranged opposite to each other, wherein each of the connecting lines between the liquid-level light transmitters 823 and liquid-level light receivers 824 passes through the central axis of intravenous infusion set 100.

In one of exemplary embodiments under working conditions, the second light beams 400, emitted from the liquid-level light transmitters 823, are received directly by the liquid-level light receiver 824 when none of the second light beams 400 passes through liquid so that the liquid-level light receiver generates a second sensing signal in response to receiving the second light beams 400. In the other hand, the second light beams 400 are not received by the liquid-level light receiver 824 when the second light beams 400 enter and pass through the liquid in the intravenous infusion set.

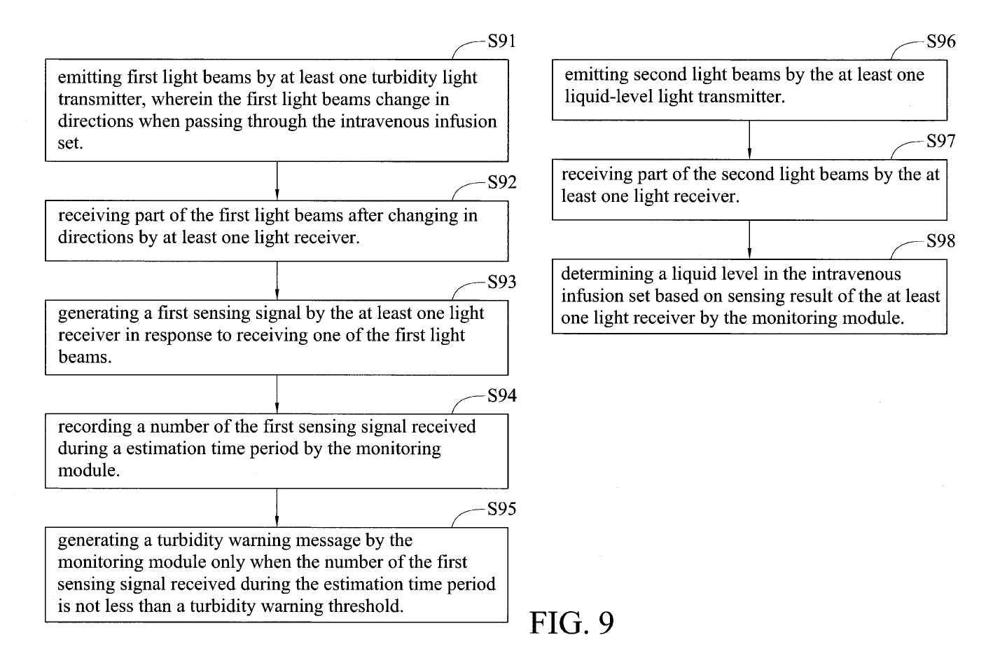

FIG. 9 illustrates a flow chart of a monitoring drug method for intravenous infusion, according to an exemplary embodiment of the disclosure. Referring to FIG. 9, the monitoring drug method for intravenous infusion may be applied to a drug monitoring device assembled outside an intravenous infusion set. Step S91 may include emitting first light beams into the intravenous infusion set by at least one turbidity light transmitter. The first light beams change in directions when passing through the intravenous infusion set. That is to say, the first light beams are reflected or refracted when there are turbid particles in the intravenous infusion set.

Step S92 may include receiving part of the first light beams after changing in directions by at least one light receiver. Step S93 may include generating a first sensing signal by the at least one light receiver in response to receiving one of the first light beams. The at least one light receiver may be arranged at a monitoring angle with respect to the at least one turbidity light transmitter so that the at least one light receiver may receive the first light beams which change in directions only within certain an angle range.

Step 94 may include recording the number of the first sensing signals received during an estimation time period by the monitoring module. Step 95 may include generating a turbidity warning message by the monitoring module when the number of the first sensing signals received during the estimation time period is not less than a turbidity warning threshold.

The higher the turbidity in the intravenous infusion set is, the more amount of the first light beams change in directions, so when the monitoring module receive the first sensing signal and the number of the first sensing signal received during the estimation time period (for example, 1 second) exceeds a standard value, that is, the turbidity warning threshold, it means that the turbidity in the intravenous infusion set is getting higher and then the monitoring module alarms people around by the turbidity warning message.

In this exemplary embodiment, the monitoring drug method for intravenous infusion further monitors a liquid level in the intravenous infusion set. Step S96 may include emitting second light beams into the intravenous infusion set by the at least one liquid-level light transmitter. Step S97 may include receiving part of the second light beams by the at least one light receiver. That is, the at least one light receiver may receive both the first light beams and the second light beams.

Step S98 may include determining the liquid level in the intravenous infusion set by the monitoring module based on a sensing result of the at least one light receiver. The monitoring module determines that there is no liquid of a certain height in the intravenous infusion set when receiving a second sensing signal corresponding to the certain height. In the other hand, determines that there is liquid of a certain height in the intravenous infusion set when without receiving any second sensing signal corresponding to the certain height. In one of exemplary embodiments, the monitoring module further compares the liquid level with a liquid-level threshold, and generates a liquid-level warning message to alarm people around when the liquid level is lower than the liquid-level threshold.

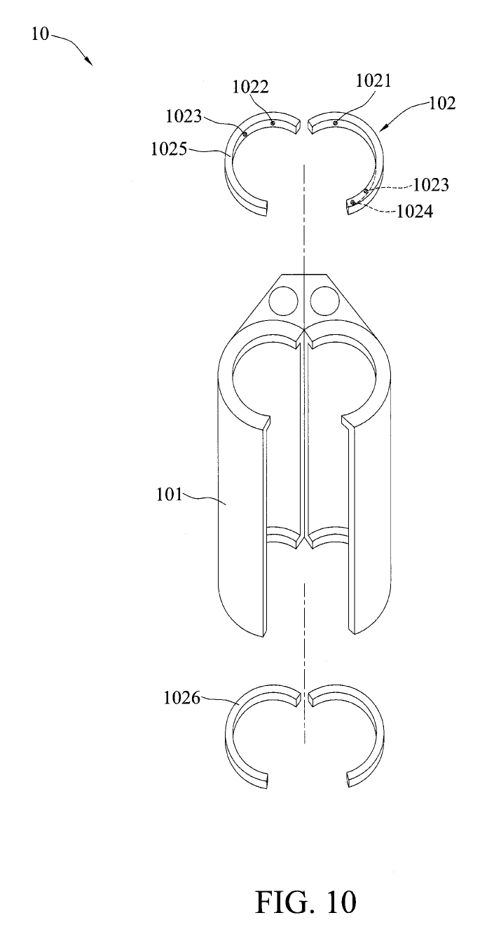

FIG. 10 illustrates another exemplary embodiment of a monitoring sensor 102 of a drug monitoring device 10 for intravenous infusion. The monitoring sensor 102 comprises a first ring 1025, a second ring 1026, at least one turbidity light transmitter 1021, at least one light receivers 1022, at least one liquid-level light transmitter 1023 and at least one liquid-level light receiver 1024. The first and second rings 1025 and 1026 are both mounted on and surrounded by a fastening assembly 101. The first ring 1025 is moveable, but the second ring 1026 is fixed. The at least one turbidity light transmitter 1021, the at least one light receivers 1022, the at least one liquid-level light transmitter 1023 and the at least one liquid-level light receiver 1024 are mounted on the first ring 1025 to vertically scan the intravenous infusion set to steadily monitor the turbidity and the liquid level by moving up and down with the first ring 1025.

In view of the aforementioned descriptions, exemplary embodiments of the present disclosure, adapted to an intravenous infusion set, may monitor both the turbidity and the liquid level and alarm the nursing people around to decrease medical risks.

It will be apparent to those skilled in the art that various modifications and variations can be made to the present disclosure. It is intended that the specification and examples be considered as exemplary embodiments only, with a scope of the disclosure being indicated by the following claims and their equivalents.

What is claimed is:

1. A drug monitoring device configured for being assembled outside an intravenous infusion set for intravenous infusion, comprising:
a fastening assembly configured for being assembled on the intravenous infusion set;
a monitoring sensor mounted on the fastening assembly, and comprising at least one turbidity light transmitter configured to emit first light beams and at least one light receiver, wherein the first light beams change in directions when passing through the intravenous infusion set, and the at least one light receiver is configured to receive a part of the first light beams changed in directions and generate a first sensing signal in response to receiving one of the first light beams; and
a monitoring module configured to receive the first sensing signal and generate a turbidity warning message when a number of the first sensing signals received during an estimation time period is greater than a turbidity warning threshold.

2. The drug monitoring device of claim 1, wherein the at least one turbidity light transmitter is arranged in a line, and the at least one light receiver correspondingly arranged in the line with respect to the at least one turbidity light transmitter.

3. The drug monitoring device of claim 1, wherein the at least one light receiver is arranged at a monitoring angle with respect to the at least one turbidity light transmitter.

4. The drug monitoring device of claim 1, wherein the monitoring sensor further comprises at least one liquid-level light transmitter mounted on the fastening assembly.

5. The drug monitoring device of claim 4, wherein the at least one liquid-level light transmitter is configured to emit second light beams, and a part of the second light beams after passing through the intravenous infusion set are received by the at least one light receiver.

6. The drug monitoring device of claim 5, wherein the monitoring module is configured to determine a liquid level in the intravenous infusion set based on a sensing result of the at least one light receiver.

7. The drug monitoring device of claim 6, wherein the monitoring module is further configured to generate a liquid-level warning message when the liquid level is lower than a liquid-level threshold.

8. The drug monitoring device of claim 4, wherein the monitoring sensor further comprises at least one liquid-level light receiver mounted on the fastening assembly.

9. The drug monitoring device of claim 8, wherein the at least one liquid-level light transmitter is configured to emit second light beams, and a part of the second light beams after passing through the intravenous infusion set are received by the at least one liquid-level light receiver.

10. The drug monitoring device of claim 9, wherein the monitoring module is further configured to determine a liquid level in the intravenous infusion set based on a sensing result of the at least one liquid-level light receiver.

11. The drug monitoring device of claim 10, wherein the monitoring module is further configured to generate a liquid-level warning message when the liquid level is lower than a liquid-level threshold.

12. The drug monitoring device of claim 1, wherein the fastening assembly comprises a first secured portion, a second secured portion coupled to the first secured portion, and a spring element clamped between the first secured portion and the second secured portion for the first secured portion and the second secured portion to elastically clamp each other around the intravenous infusion set.

13. A method for drug monitoring in intravenous infusion, comprising:
emitting first light beams by at least one turbidity light transmitter with the first light beams changing in directions when passing through an intravenous infusion set;
receiving a part of the first light beams by at least one light receiver after the first light beams changing in directions;
generating a first sensing signal by the at least one light receiver in response to receiving one of the first light beams;
recording a number of the first sensing signals received by a monitoring module during an estimation time period; and
generating a turbidity warning message by the monitoring module when the number of the first sensing signals received during the estimation time period is greater than a turbidity warning threshold.

14. The method of claim 13, wherein the at least one light receiver is arranged at a monitoring angle with respect to the at least one turbidity light transmitter.

15. The method of claim 13, further comprising:
emitting second light beams into the intravenous infusion set by at least one liquid-level light transmitter;
receiving a part of the second light beams by the at least one light receiver; and
determining a liquid level in the intravenous infusion set by the monitoring module based on a sensing result of the at least one light receiver.

16. The method of claim 15, further comprising:
generating a liquid-level warning message by the monitoring module when the liquid level is lower than a liquid-level threshold.

17. The method of claim 13, further comprising:
emitting second light beams into the intravenous infusion set by at least one liquid-level light transmitter;
receiving a part of the second light beams by at least one liquid-level light receiver; and
determining a liquid level in the intravenous infusion set by the monitoring module based on a sensing result of the at least one liquid-level light receiver.

18. The method of claim 17, further comprising:
generating a liquid-level warning message by the monitoring module when the liquid level is lower than a liquid-level threshold.
